(19) United States
(12) Patent Application Publication
(10) Pub. No.: US 2018/0313818 A1
(43) Pub. Date: Nov. 1, 2018

(54) REMOTE SOBRIETY MONITORING SYSTEMS, DEVICES AND METHODS

(71) Applicant: SOBERLINK HEALTHCARE LLC, Huntington Beach, CA (US)

(72) Inventors: Brad Keays, Manhattan Beach, CA (US); Christopher J. Pursley, Fullerton, CA (US); Daniel Rhodes, San Diego, CA (US); Casey Hanrahan, Fullerton, CA (US)

(21) Appl. No.: 15/791,282

(22) Filed: Oct. 23, 2017

Related U.S. Application Data
(63) Continuation of application No. 14/963,187, filed on Dec. 8, 2015, now abandoned.
(60) Provisional application No. 62/089,140, filed on Dec. 8, 2014.

(51) Int. Cl. G01N 33/497

(52) U.S. Cl. CPC G01N 33/4972

(57) ABSTRACT

Systems, methods and devices are disclosed for monitoring sobriety of a first user by a second user which can be discrete, wirelessly linked via a communications network and can provide various forms of alerts to the first and second users.

### CROSS REFERENCE TO RELATED APPLICATIONS

This application is a continuation of U.S. patent application Ser. No. 14/963,187, filed Dec. 8, 2015, which claims priority to U.S. Provisional Application No. 62/089,140, entitled “SOBRIETY MONITORING SYSTEM”, filed Dec. 8, 2014, which are both hereby incorporated in their entireties by reference.

### FIELD OF THE INVENTION

This disclosure relates generally to systems, device and methods for remote sobriety monitoring.

### BACKGROUND OF THE INVENTION

Recovering alcoholics or other substance abusers may benefit from the supervision of a sober chaperone such as a sober buddy, sober companion or sober coach to assist a recovering alcoholic in maintaining abstinence from alcohol outside of a treatment facility. Such a sober companion commonly chaperones the recovering alcoholic or substance abuser on a constant basis, or may be available on an on-call basis to accompany a recovering alcoholic or substance abuser periodically or as needed during certain activities. Such supervisory care can be quite expensive, which may have the unfortunate consequence of reducing or eliminating the services of such supervisory care.

People struggling with alcohol often conceal their abuse, making it difficult for concerned family members to confirm their suspicions and intervene. Because alcohol leaves the system quickly, it is important to test for alcohol consumption by using a breathalyzer or another similar alcohol testing method. Confirmation of a drinking problem becomes increasingly difficult during periods when testing for alcohol consumption is not easily enforced, such as during travel for business or college, for example. It would be useful to provide a method for parents to be able to monitor alcohol use anywhere by their children, and for spouses to monitor alcohol use anywhere by their spouses, in order to eliminate suspicions and confirm whether the family member has a drinking problem. It would also be useful to provide a method for companies to deter alcohol abuse by employees during work hours. Industries that rely heavily on driving and have limited employee supervision could also benefit from a method allowing the monitoring of alcohol use by employees as a way to confirm employee sobriety during work hours. Although drug testing is common in the workplace, since alcohol is metabolized relatively quickly, and is not easily tested, it would also be useful to provide a method for immediate confirmation of an employee's alcohol level at any given time.

Court ordered sobriety is also commonly required as a condition of probation or other court imposed rehabilitative or behavior altering programs. Reporting to a stationary facility, one's probation officer, or even one's home in order to be tested for substance use is often an embarrassing and time consuming ordeal that does not facilitate healthy reintegration into society. Thus, the discrete remote monitoring of a person under such a program by the court, or other authority, without requiring the monitored person to excuse themselves from society for more than a brief period of time would be useful in reintegrating the monitored person into society without the awkward and embarrassing effects of traditional monitoring procedures. Such a system is also useful to provide a system of monitoring where those monitored are emboldened to no longer feel like societal outcasts and are thus increasingly motivated to maintain their sobriety.

It would therefore be desirable to provide methods, devices and systems of providing supervisory monitoring of sobriety that are discrete, portable, tamper-proof, and effective, and that can automatically alert a monitoring station of the need for attention and possible corrective or medical action by such a supervisory sober buddy or sober companion on an on-call basis. The present invention meets these and other needs.

### SUMMARY OF THE INVENTION

Briefly, and in general terms, the present invention provides for systems, devices and methods for monitoring sobriety of a user, utilizing a portable, handheld breath testing device transmitting breath test results to a monitoring station via a wireless network. The breath test data is stored by the monitoring station and is retrievable therefrom by a monitor, such as a parent, guardian, parole officer, court liaison, spouse, sobriety coach, friend, sobriety monitoring company, or other authorized group or individual. In this manner, the monitor is able to respond appropriately to the detected utilization of alcohol and/or other substances by a user.

By using the methods, devices and system of the present invention, a family member trying to build back trust in family relationships can prove that they are making behavior changes by sending breath test reports on a predetermined schedule, or when randomly requested by the family. The present invention helps a person prove that they are making healthier choices in life and making steps toward rebuilding trust in family relationships. Families can benefit from knowing that loved ones are sober enough to drive, and the present invention can be used remotely to determine a person's sobriety or that blood alcohol levels are in an acceptable range. For families who want to monitor their children or spouses, the sobriety monitoring system of the present invention can send a breath test report directly to a mobile device such as a smartphone or tablet.

The present invention can also be used for immediate confirmation of an employee's alcohol level at any given time. Particularly those companies with employees who drive as a part of their employment would benefit by keeping their employees sober during working hours. The present invention also can be used in rehabilitative aftercare, and can be used to monitor multiple patients, and the present invention can be used by a sober companion during times when they are not able to accompany one or more of the patients.

Due to the compact, handheld portable nature of the present invention, the present invention mitigates the social stigma, personal embarrassment and general inconvenience typically associated with traditional sobriety monitoring techniques, e.g. ankle bracelets, urinalysis, fixed testing sites, etc.

These and other aspects and advantages of the invention will be apparent from the following detailed description and the accompanying drawing, which illustrates by way of example the features of the invention.

### BRIEF DESCRIPTION OF THE DRAWING(S)

Illustrated in the accompanying drawing(s) is at least one of the best mode embodiments of the present invention. In such drawing(s):

FIG. 1A is a schematic diagram of an example embodiment of a sobriety monitoring system in accordance with the present invention;

FIG. 1B is an example embodiment of a network connected server system in accordance with the present invention;

FIG. 1C is an example embodiment of a user device in accordance with the present invention;

FIG. 2 is a schematic diagram of an example embodiment of a breath testing device in accordance with the present invention;

FIG. 3 is an example embodiment of a web portal in accordance with the present invention;

FIG. 4 is a perspective view of an example embodiment of a breath testing device in accordance with the present invention;

FIG. 5 is an example embodiment of a report in accordance the present invention;

FIGS. 6A-6B are example embodiments of web portals in accordance with the present invention;

FIGS. 7A-7C are example embodiments of web portals in accordance with the present invention;

FIG. 8 is an example embodiment of a report in accordance with the present invention;

FIG. 9 is a schematic of an example embodiment of a breath intake of a breath monitoring device in accordance with the present invention;

FIG. 10 is a flowchart of an example embodiment of a counter process in accordance with the present invention;

FIG. 11 is a schematic diagram of an example embodiment of a breath testing device utilizing personal-area-network connectivity in accordance with the present invention; and

FIG. 12 is a front elevated view of an example embodiment of a portable calibration unit according to the present invention.

### DETAILED DESCRIPTION OF THE PREFERRED EMBODIMENTS

The above described drawing figures illustrate the invention in at least one preferred, best mode embodiment, which is further defined in detail in the following description. Those having ordinary skill in the art may be able to make alterations and modifications to what is described herein without departing from its spirit and scope. Therefore, it should be understood that what is illustrated is set forth only for the purposes of example and should not be taken as a limitation on the scope of the present system and method.

Described now in detail is a method and system for monitoring sobriety of a user according to at least one preferred embodiment.

### Basic Structure

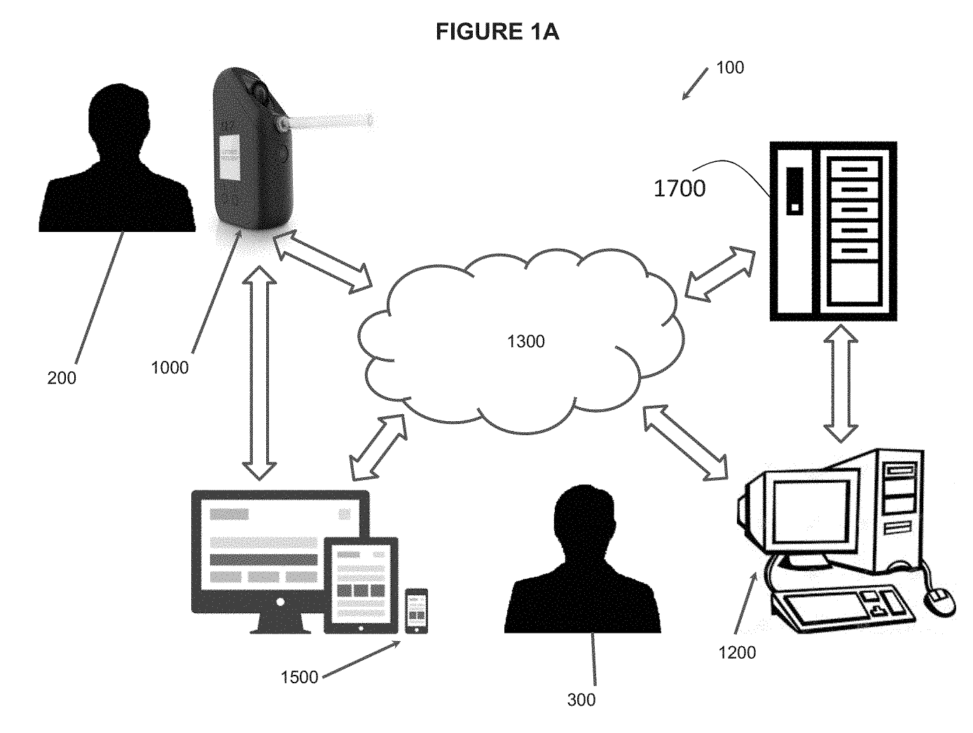

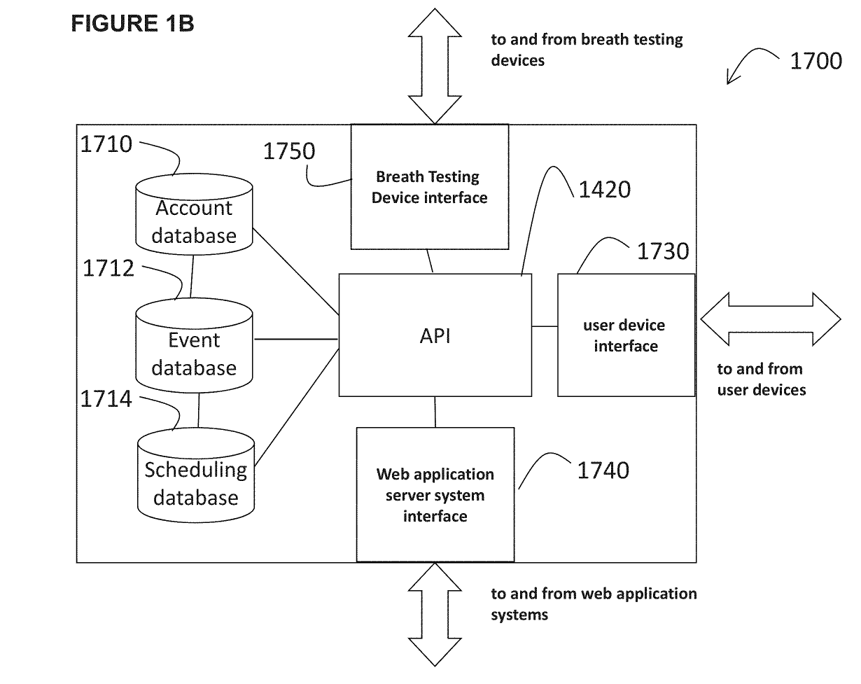

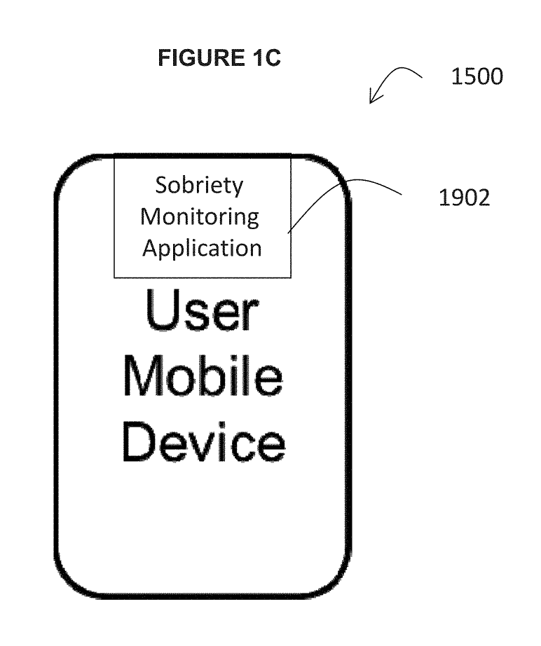

As shown in FIG. 1A, a schematic diagram of an example embodiment of a sobriety monitoring system 100 can include: a portable, handheld breath testing device 1000 communicatively coupled to a monitoring station 1200 and server 1700 via a wireless network 1300. The breath testing device 1000 tests the breath of a user 200 for the presence of alcohol and/or other substances. The breath testing device 1000 generates breath test data in response to assessment of the user's breath, the breath test data can indicate whether the user 200 has recently utilized alcohol and/or other substances. The breath test data can be wirelessly transmitted by the breath testing device 1000 via the network 1300 to the monitoring station 1200 and the server 1700, which may be any device or system at a location where the breath test data is received, including by way of non-limiting example: a cellular/smart phone, an email account, a website, a network database, and a memory device. The breath test data is stored by the monitoring station 1200, the server 1700 or both, and is retrievable therefrom by a monitor 300, such as a parent, guardian, parole officer, court liaison, spouse, sobriety coach, friend, sobriety monitoring company, or other authorized group, individual or combination thereof. In this manner, monitor 300 is able to respond appropriately to the detected utilization of alcohol and/or other substances by the user 200. Preferably, the monitor 300 is able to retrieve the breath test data via a network connected user interface device 1500, communicatively coupled—via the network 1300—to the monitoring station 1200, the server 1700 and/or to the breath testing device 1000.

Server 1700 can include applications distributed on one or more physical servers, each having one or more processors, memory banks, operating systems, input/output interfaces, and network interfaces, all known in the art. A plurality of user interface devices 1500 can be communicatively coupled to network 1300 such as a public network (e.g. the Internet and/or a cellular-based wireless network, or other network), a private network or a combination thereof. User interface devices 1500 and monitoring stations 1200 can include for example mobile devices (e.g. phones, tablets, or others) desktop or laptop devices, wearable devices (e.g. watches, bracelets, glasses, etc.), other devices with computing capability and network interfaces and so on. Sobriety monitoring system architecture 100 can include a network connected server system 1700 which can include hosting and/or interfacing, by way of physical servers, websites, webpages, web applications, social media platforms, advertising platforms, and others.

FIG. 1B shows an example embodiment of a diagram of a server system 1700 according to the invention including at least one user device interface 1730 implemented with technology known in the art for communication with user devices (e.g. user devices 1500 of FIG. 1A). The server system 1700 can also include at least one web application server system interface 1740 for communication with web applications, websites, webpages, websites, social media platforms, and others over a network. The server system 1700 can further include an application program interface (API) 1720 that is coupled to an account database 1710, event database 1712, and scheduling database 1714 and can communicate with interfaces such as the user device interface 1730, breath testing device interface 1750 and web application server system interface 1740, or others. The API 1720 can instruct the databases 1710, 1712, 1714 to store (and retrieve from the databases) information such as user account information, associated account information, event information or others as appropriate. The databases 1710, 1712, 1714 can be implemented with technology known in the art such as relational databases and/or object oriented databases or others.

FIG. 1C shows a diagram of a user device 1500 according to an embodiment of the invention that includes a network connected sobriety monitoring application 1902 that is installed in, pushed to, or downloaded to the user mobile device 1500. In many embodiments user mobile devices 1500 are touch screen devices such as smart phones or tablets. User mobile devices 1500 are implemented with memory, processors, communications links, power supplies such as rechargeable batteries, interfaces such as screens displaying GUI's, buttons, touchpads, software stored in memory and executed by processors, audio input and output components, video input and output components, and others. Software can include computer readable instructions stored on non-transitory computer readable media such as computer memory.

In some embodiments, a server supported website comprises a mobile website accessible via a sobriety monitoring application 1902 on a personal computing device 1500 such as a mobile device (e.g. smart phone). The mobile website may be a modified version of the server supported website with limited or additional capabilities suited for mobile sobriety monitoring by monitor 300. In some embodiments no specific application 1902 is required but users can access a sobriety monitoring system through a web browser of the user mobile device 1500.

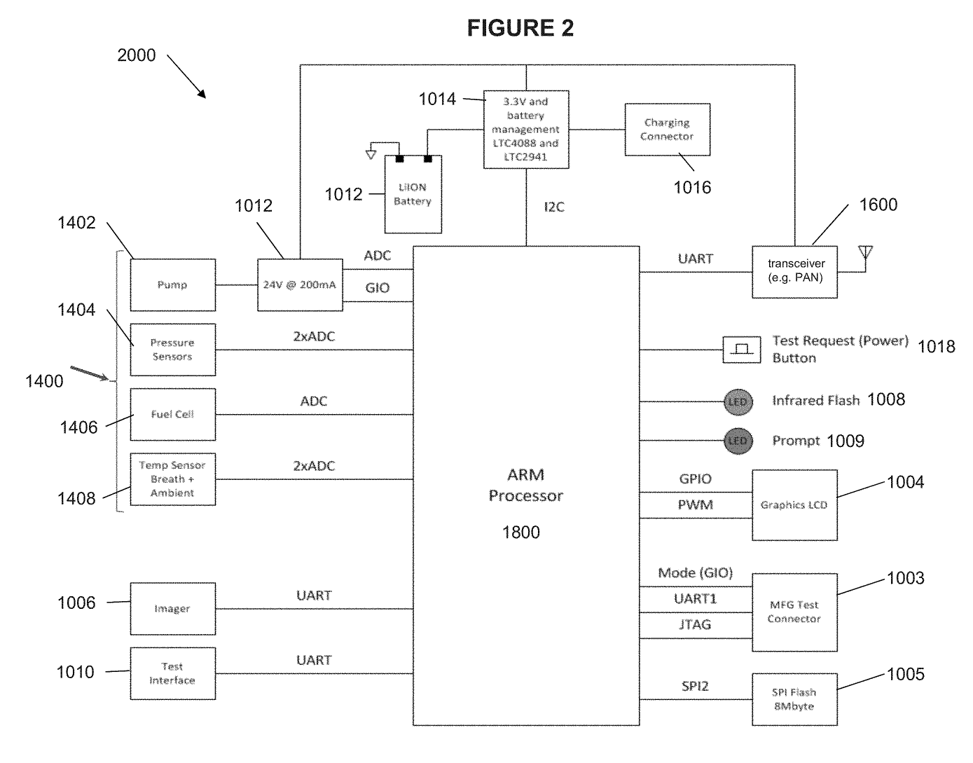

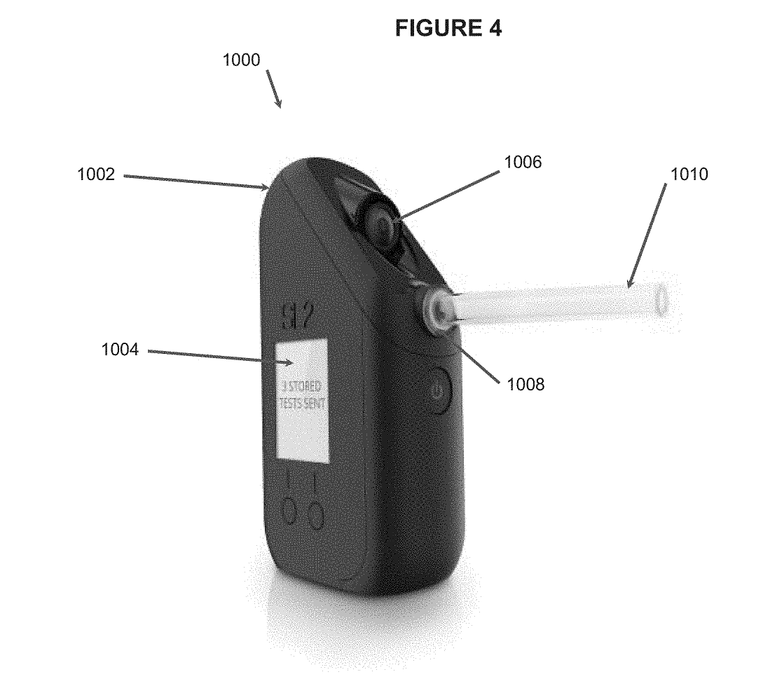

As shown in the example embodiment of FIG. 2, a breath testing device 1000 may comprise a memory 1005, such as a SPI FLASH 8 MB memory, communicatively coupled to a breath testing module 1400. Breath testing module 1400 is operable to measure the user's breath and convert the measurements into breath test data, as will be described elsewhere herein and breath testing module 1400 can be communicatively coupled to a wireless transceiver 1600, such as a personal area network (PAN). Each of memory 1005, breath testing module 1400 and transceiver 1600 can be communicatively coupled to a control unit 1800, such as an ARM processor, for controlling the operations thereof in accordance with the functionalities described herein. As breath testing device 1000 is portable and handheld, each of the components are preferably located within, immediately adjacent to, or exposed within and/or without, a device housing (e.g. housing 1002 of FIG. 4) whose dimensions are such that the breath testing device 1000—as a whole—may be discretely carried by the user, for example, within a pocket or small purse.

Further, as shown for example in the breath testing device architecture 2000 of FIG. 2, corresponding to a breath testing device 1000, may comprise: a central processing unit 1800 communicatively coupled to: a pump 1402, one or more pressure sensors 1404, a fuel cell 1406, and one or more temperature sensors 1408, which together can be included in a breath testing module 1400. An imager 1006, a test interface 1010, a flash memory 1005, a test connector 1003, a display 1004, an LED prompt 1009, a flash LED 1008, a test request/power button 1018, a wireless transceiver 1600, batteries 1012 and 1014, and associated charging interface 1016 can also be communicatively coupled to central processing unit 1800. Pump 1402 can also be connected through a converter 1012 to transceiver 1600. Each of these components are described further herein in the context of their functionalities within the described embodiments.

In an example embodiment, a monitoring station 1200, as shown for example in FIG. 1A, can comprise a server system 1700, hosting a server supported website (not shown). Server supported website can be supported by a server system 1700 comprising one or more physical servers, each having a processor, a memory, an operating system, input/output interfaces, and network interfaces, all known in the art, coupled to the network 1300. The server supported website can comprise one or more interactive web portals through which a monitor 300 may monitor the sobriety of a user 200 using a breath monitoring device 1000, in accordance with the described embodiments. In particular, the interactive web portals may enable the monitor 300 to retrieve breath test reports generated using data from the breath testing devices 1000 of one or more users (e.g. user 200), set or modify breath test schedules, and/or set or modify preferences, as described herein. Preferably, the interactive web portals are accessible via a personal computing device 1500, such as for example, a home computer, laptop, tablet, and/or smart phone.

Additional details of these features and others may be found in U.S. Pat. No. 8,707,758, issued on Apr. 29, 2014; U.S. Pat. No. 8,381,573, issued on Sep. 15, 2010; and U.S. application Ser. No. 13/274,553, filed on Oct. 17, 2011, the entire disclosures and contents of which are herein incorporated by reference in their entirety. Additional details of these features and others may also be found in the Appendix hereto filed herewith, the entire disclosure and content of which is herein incorporated by reference in its entirety.

Scheduling

In order to more effectively monitor a user 200 for sobriety, in some embodiments a breath testing device 1000 preferably receives and tests the user's breath in accordance with a schedule. The schedule may be generated and/or stored by the server system 1700 in a scheduling database 1714 based on input provided by the monitor 300. The schedule may be pushed to or accessed by the breath testing device 1000 so as to prompt the user 200 to initiate scheduled breath tests. When breath test data associated with a scheduled breath test is not received by monitoring station 1200 in accordance with the schedule, the lack of scheduled breath test may be associated with a ‘missed’ result of the breath test.

### Test Period

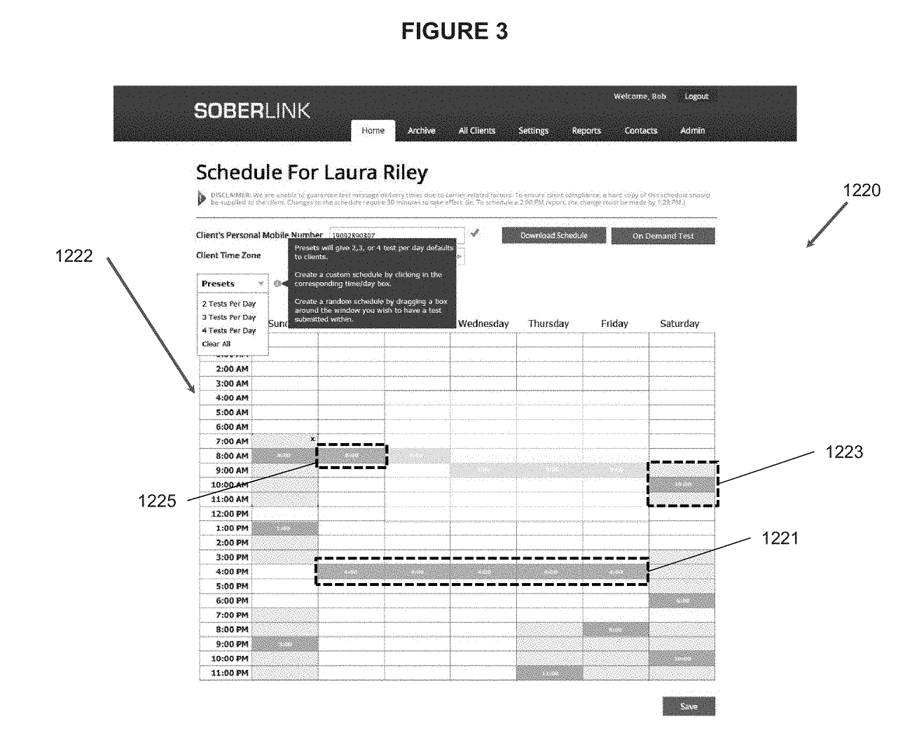

In an example embodiment, the schedule may be accessed and/or manipulated by the monitor 300 via the server supported website. FIG. 3 reflects an example embodiment of an interactive web portal 1220 for a monitor 300 to generate and/or modify the schedule 1222 as displayed on a display of a user device (e.g. devices 1500 of FIG. 1A). As reflected in FIG. 3, the schedule may consist of predetermined test periods 1221, random test periods 1223, and/or on-demand test periods 1225. The monitor 300 sets a predetermined test period 1221 by selecting a single or multiple date/time options for the test to occur. The monitor 300 may set multiple predetermined test periods 1221 by selecting a plurality of single dates/times for the test to occur, as shown by the Monday-Friday 4:00 pm timeslots of FIG. 3. In some embodiments, the system provides for selection of pre-constructed testing using testing programs stored in memory.

The monitor 300 can set a random test period 1223 by selecting a single date/time option or a continuous range of date/time options—reflecting the temporal bounds within which the monitor 300 desires the breath test to randomly occur—as well as selecting the number of breath tests the monitor desires to be taken during the random test period. As the selection includes or consists of a range, it may be ‘resized’ according to the preference of the monitor 300. The server system 1700 then randomly schedules the desired number of tests to occur during the set random test period 1223. Preferably, if the generated schedule is a periodic schedule (e.g. weekly, bi-weekly, monthly, etc.), the randomly generated test periods 1223 are re-randomized within each set random test period for each successive schedule cycle. In the example embodiment, a period from 9:00 am-12:00 pm has been selected.

In addition, or as an alternative, the monitor 300 may also select an on-demand test 1225, reflecting a desire to schedule an immediate breath test (or as closely thereto as practical). Preferably, on-demand tests 1225 are not recycled to the next schedule cycle.

### Test Window

Additionally, for each scheduled breath test, there may exist a test window, i.e. a period of time from the inception of the test period during which the scheduled breath test can be taken by the user before the breath test is considered ‘missed’ by the system. The test window may be a default test window, or may be generated or otherwise modified by the monitor 300, preferably via the server supported website. The monitor 300 may select from a plurality of predetermined options for the test window, including for example, 30 min, 60 min, 120 min, 180 min, 240 min, or custom duration test windows. In some embodiments, the monitor 300 may assign unique test windows to one or more of the test periods. In some embodiments, the test window may not exceed a predetermined duration. Additionally, presets can allow these windows to occur multiple times per day, week, or month.

### Conflict Check

In some embodiments, the system may perform a check for ‘conflicts’ between the scheduled test periods and test windows. For example, if test periods are scheduled for every other hour of the day with test windows of 180 minutes, then successive test periods would overlap with the test windows of the prior test period. This can be an undesirable result, as it may encourage users to perform a single breath test (or closely successive breath tests) during the overlapping period, so as to provide the user with more time before the next scheduled breath test in which to imbibe alcohol and more time in which it could be metabolized by the user without triggering an alert. This undesirable result may also occur even with merely abutting—rather than overlapping—test periods/windows. Consequently, in some embodiments, a ‘conflict’ may be determined where there is an insufficient buffer period between scheduled breath tests such that the temptation to drink alcohol is not sufficiently mitigated.

Preferably in some embodiments, when a ‘conflict’ is detected, the system displays a prompt to the monitor 300 identifying the conflict as well as optional monitor actions to resolve the conflict. The optional monitor actions may include, for example, modifying one or more of the test periods and/or testing windows, and/or cancelling the proposed modification. Alternatively, one or more of these options may be automatically executed without monitor action, input or confirmation

### User Alerts

As discussed herein, the schedule may be accessed by the breath testing device 1000, and the breath testing device 1000 may prompt a user 200 to initiate scheduled breath tests based on the schedule. The prompt may be one or more of: a visual prompt, an audio prompt, and a tactile prompt. Accordingly, as shown in FIG. 4, the breath testing device 1000 may comprise: a display screen 1004 for displaying a visual prompt, an audio speaker (not shown) for playing an audio prompt, and a vibrator (not shown) for producing a tactile prompt. These prompts are varied in possible nature and should be understood to those of skill in the art as being performed by the components which are operatively coupled to one or more processing units. Each of the display screen 1004, audio speaker and vibrator can be communicatively coupled to non-transitory memory storing instructions for functions or operations thereof and a control unit including one or more processors for controlling the functions and operations by executing the stored instructions. The visual prompt may include text, images or a combination thereof, or a series of such visual prompts or combinations of visual prompts displayed on display screen 1004. An audio prompt may include one or more different audio prompts, or a series thereof, such as sounds or language based statements or instructions. Each prompt may be stored in the memory and retrieved in accordance with the schedule using, for instance, one or more timers or time monitoring elements (not shown).

In some embodiments, a prompt can be an e-mail, text message or both generated by a monitoring station 1200, server 1700 or otherwise (e.g. the server supported website), or a combination thereof and transmitted to a stored e-mail account or cellular phone of the user 200, monitor 300 or both. In some embodiments—particularly in those embodiments utilizing a mobile website, mobile software application (“app”) or combination of both described herein—the prompt may comprise a ‘post’ on the user's ‘wall,’ ‘feed,’ or otherwise or a message delivered to a private messaging interface of the monitor 300. In some embodiments, the prompt may comprise an automated or live phone call to the user 200, monitor 300, or both. As such, prompts can generally be generated by the breath monitoring device 1000 itself or by server system 1700.

Additional details of these features and others may be found in U.S. Pat. No. 8,707,758, issued on Apr. 29, 2014; U.S. Pat. No. 8,381,573, issued on Sep. 15, 2010; and U.S. application Ser. No. 13/274,553, filed on Oct. 17, 2011, the entire disclosures and contents of which are herein incorporated by reference in their entirety. Additional details of these features and others may also be found in the Appendix hereto filed herewith, the entire disclosure and content of which is herein incorporated by reference in its entirety.

### Testing

As discussed herein with reference to FIG. 1, the breath testing device 1000 tests the breath of the user 200 for the presence of alcohol and/or other substances, generating breath test data in response to receiving the user's breath and analyzing it. The breath test data is then transmitted to the monitoring station 1200, server 1700, user device 1500, or a combination thereof, preferably via wireless transmission over the network 1300.

During a breath test, the breath testing device 1000 receives a user's breath, analyzes the user's breath and converts this information into breath test data to be transmitted to the monitoring station 1200, server 1700, user device 1500, or a combination thereof. The breath test data preferably reflects the blood alcohol content (“BAC”) of the user 200 during the breath test, or an indication of whether the user's BAC is above or below a certain BAC threshold. The BAC threshold may be set and modified by the monitor 300 via the server supported website.

Additionally, the breath test data may be displayed on the breath testing device 1000 via the display screen 1004 located exterior to the device housing 1002, as shown in FIG. 4, for example. Alternatively, or in addition, a visual prompt may be generated based on the breath test data and displayed on the display screen 1004 of the breath testing device 1000, the visual prompt indicating a ‘pass,’ ‘fail,’ ‘missed,’ or ‘inconclusive’ result of the breath test. Moreover, in the event of either a ‘fail’ or ‘inconclusive’ breath test, the visual prompt may also instruct the user 200 to re-administer a breath test.

Additional details of these features and others may be found in U.S. Pat. No. 8,707,758, issued on Apr. 29, 2014; U.S. Pat. No. 8,381,573, issued on Sep. 15, 2010; and U.S. application Ser. No. 13/274,553, filed on Oct. 17, 2011, the entire disclosures and contents of which are herein incorporated by reference in their entirety. Additional details of these features and others may also be found in the Appendix hereto filed herewith, the entire disclosure and content of which is herein incorporated by reference in its entirety.

### Queueing

In some instances, a breath testing device 1000 may be temporarily disconnected from the network 1300 and/or otherwise unable to transmit the breath test data, as described herein. In such instances, it is desirable for the breath testing device 1000 to store the breath test data from one or more administered breath tests until such time as the breath testing device regains network connectivity, and thereafter to transmit the breath test data to the monitoring station 1200, server 1700, user device 1500, or a combination thereof. In such instances, it is also desirable that the belatedly transmitted breath test data be identifiable as in accordance with a scheduled breath test.

As shown in FIG. 2, the breath testing device architecture 2000 may comprise the memory 1005 communicatively coupled to the breath testing module 1400 for converting the user's breath into breath test data, a clock (not shown) for generating timestamp data in association with the administered breath test, and a transceiver 1600, each communicatively coupled to the control unit 1800 for controlling the operations thereof in accordance with the functionalities described herein.

The timestamp data is preferably utilized to determine whether an associated breath test has been administered in accordance with the schedule. Accordingly, the timestamp data may be retrievably stored in the memory 1005 in association with the administered breath test, and/or transmitted to the monitoring station 1200 in association with the breath test data.

When the breath testing device 1000 fails to transmit the breath test data of one or more administered breath tests to the monitoring station 1200, as described herein, due to, for example, a lack of network connectivity, the breath testing device 1000 can store breath test data associated with one or more breath tests (and associated timestamps) in a queue in the memory 1005, preferably according to their associated timestamps. In such event, the breath testing device 1000 may periodically retry transmitting the breath test data (and associated timestamps) stored in the queue, such that when network connectivity is re-established the data is transmitted in accordance with the embodiments described herein.

In some embodiments, breath test data (and associated timestamps) are saved in the memory 1005 with a transmission status data indicating whether the breath test data has been successfully transmitted to the monitoring station 1200. If the breath test data has been successfully transmitted to the monitoring station 1200, as indicated by the associated transmission status data, the control unit 1800 will not retry transmitting the breath test data. Data can be updated based on confirmation or acknowledgement data sent over the network from the monitoring station 1200 or server 1700 to the breath monitoring device 1000. However, if breath test data has not been successfully transmitted to the monitoring station 1200, as indicated by the associated transmission status data, the control unit 1800 will retry transmitting the breath test data based on predetermined or pre-programmed rules, thresholds and timers. In some embodiments, the transmission status data identifies the number of unsuccessful attempts at transmitting the breath test data.

In some embodiments, the breath test data (and associated timestamp data) may be manually retrieved if necessary. Accordingly, in some embodiments, the breath testing device 1000 may further comprise a data input/output port (not shown), such as for a USB, fire-wire, or other wired data transfer, communicatively coupled to the memory 1005 and a user interface device for accessing the data stored in the memory 1005. The user interface device can include a variety of devices including and not limited to specialty devices, smart phones, laptop computers, desktop computers, and others as described herein and known in the art or later developed.

Additional details of these features and others may be found in U.S. Pat. No. 8,707,758, issued on Apr. 29, 2014; U.S. Pat. No. 8,381,573, issued on Sep. 15, 2010; and U.S. application Ser. No. 13/274,553, filed on Oct. 17, 2011, the entire disclosures and contents of which are herein incorporated by reference in their entirety. Additional details of these features and others may also be found in the Appendix hereto filed herewith, the entire disclosure and content of which is herein incorporated by reference in its entirety.

### Reporting

The breath test data (and the other types of data described herein) may be utilized by the system to generate one or more reports based thereon. Such reports may include the breath test data (and other data) from one or more administered and/or scheduled breath tests. In some embodiments, the reports are generated automatically at specified intervals, e.g. daily, weekly, monthly, per breath test.

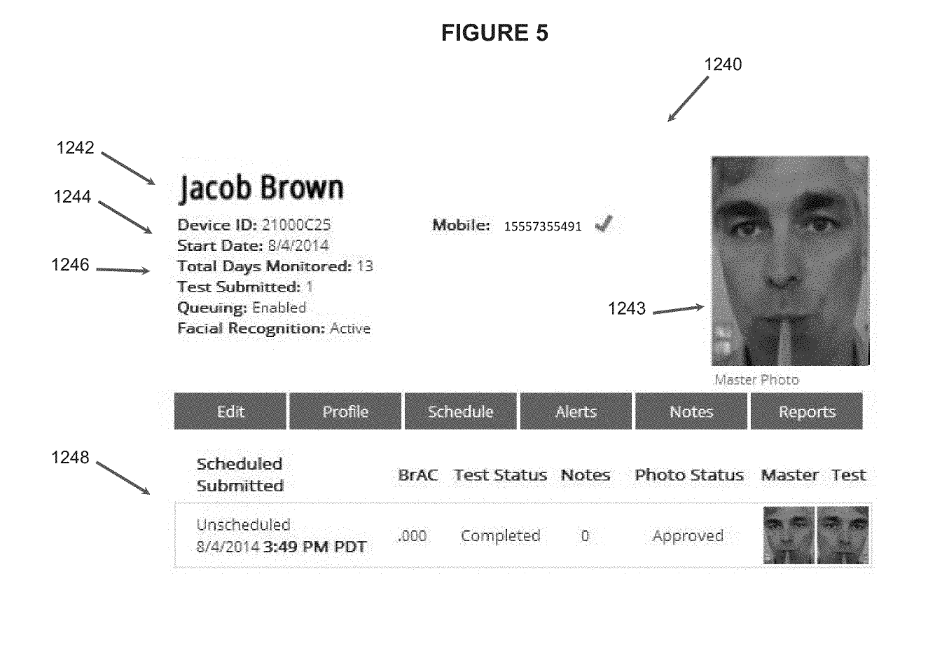

FIG. 5 reflects an example embodiment of a report 1240 generated in accordance with the embodiments described herein. The report 1240 can include: user identification data 1242, including a user name and, in some embodiments, a reference image 1243; device identification data 1244, including a unique device identification number such as a serial number or other identifier, a device mobile number; and monitoring program data 1246, including a program initiation date, a number of days monitored, a number of tests administered and/or submitted as well as information about whether various features (e.g. queueing and/or facial recognition) are active/enabled or inactive/disabled. The report 1240 can also include information on one or more administered and/or scheduled breath tests 1248, including: whether the breath test was scheduled or unscheduled, the date/time of the breath test, the BrAC (Breath Alcohol Content) level of the user's breath, the test status (such as completed or incomplete), a captured image of the user (or other user identification data such as biometric fingerprinting, or others) during the breath test, and/or whether the captured image matches, corresponds or is similar to the reference image 1243. This matching can be processed by the device 1000 itself or can be accomplished on the server 1700 or other computing devices. While the information displayed in the report 1240 of FIG. 5 is discussed herein, it should be understood that generated reports may include more or less information, as appropriate in various embodiments. In particular, it is contemplated that the information contained in the generated reports may be customized by a monitor 300 via an interactive web portal to the server supported website. It is also contemplated that the monitor 300 may customize one or more monitoring preferences not only with regards to the information provided, but as to how the reports are transmitted and/or displayed.

In some embodiments, generated reports may be accessed by the monitor 300 via an interactive web portal to the server supported website, whereby the monitor 300 may review one or more reports by accessing them online over a network 1300 (see e.g. FIG. 1). In some embodiments, the generated reports may be e-mailed, text messaged or both to an e-mail account or cellular phone of the monitor 300, user 200 or both, respectively. In some embodiments—particularly in those embodiments utilizing a mobile website and/or mobile software application (“app”) (see e.g. FIG. 1C Sobriety Monitoring Application 1902) described herein—the generated reports may comprise a ‘post’ on the user's or monitor's (or a combination of both) ‘wall,’ ‘feed,’ or otherwise.

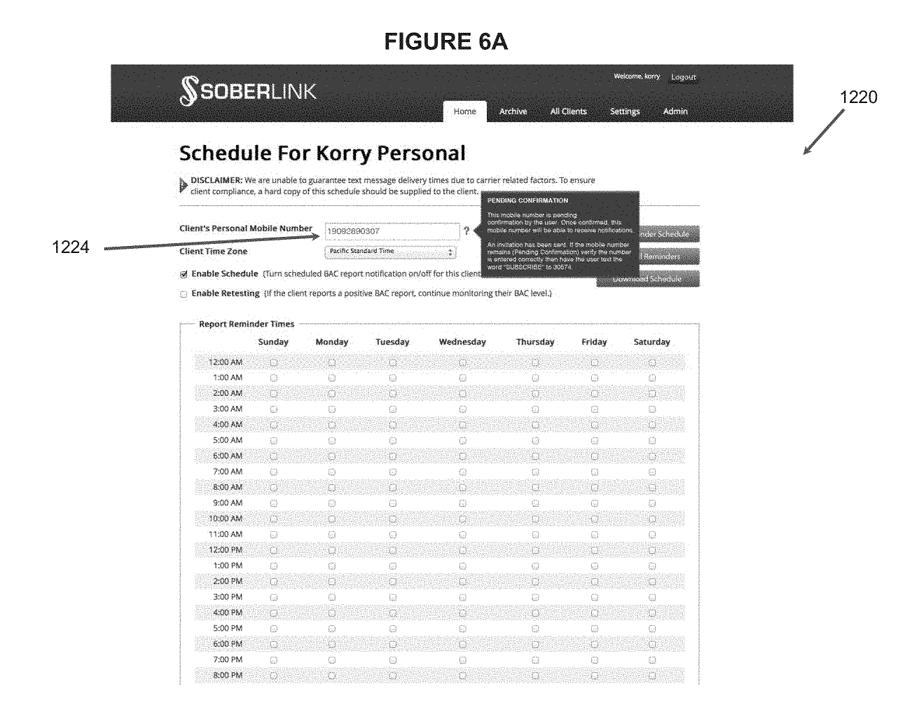

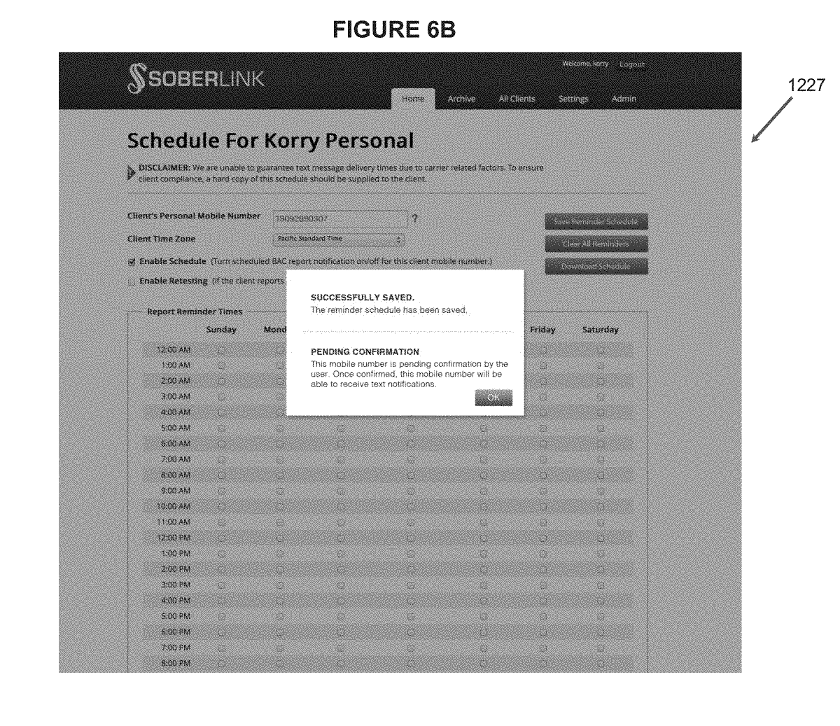

In some embodiments, the system can provide a ‘text-message’ opt-in option. When, through a registration process of creating an account, a mobile number is added to the system or otherwise updated on the website, the system identifies whether the number has previously been blacklisted or confirmed. If it has not been either blacklisted, confirmed or both, the system can transmit an invitation notification, such as a text message, to that mobile number to opt-in to receive text-message alerts and/or reports. If a monitor 300 (or user 200) responds to the invite with a “SUBSCRIBE” response, the mobile number can be marked as confirmed in memory and from that point forward is able to receive text messages from the system. At any time, the monitor 300 (or user 200) may text “STOP” to unsubscribe. FIGS. 6A-6B illustrate example embodiments of web portals 1220, 1227 respectively, whereby a monitor 300 may opt-in to receive text-message alerts and/or reports via mobile number field 1224 of web portal 1220 and a confirmation notification in web portal 1227.

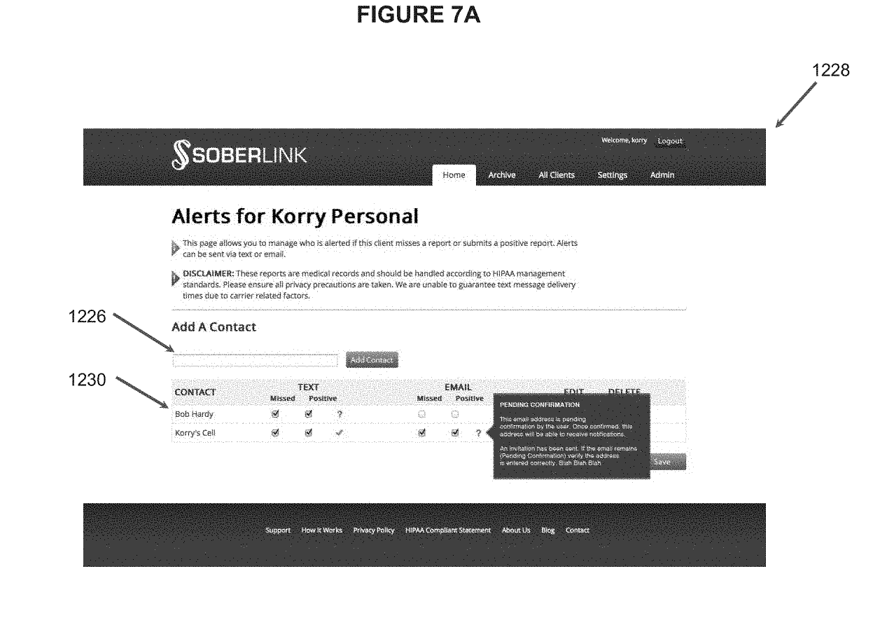

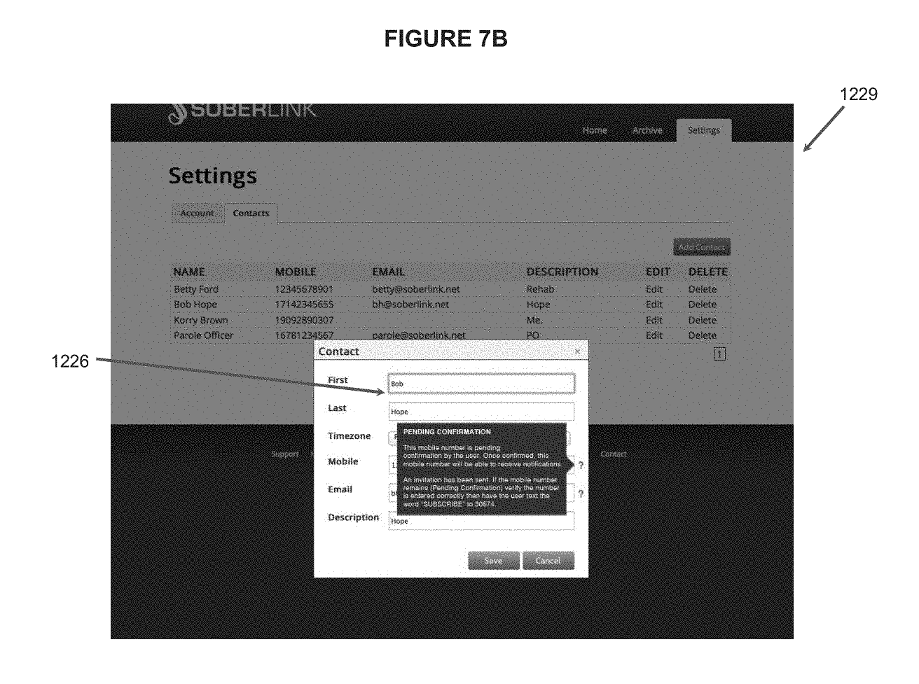

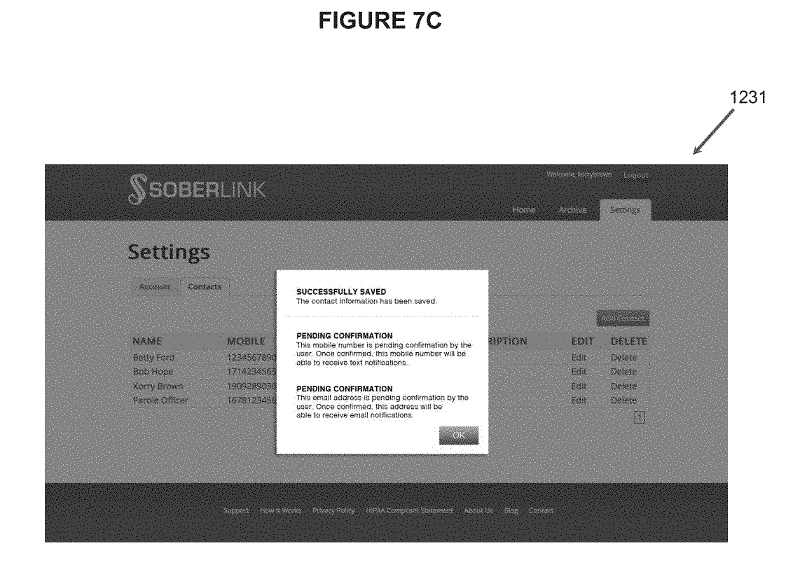

FIGS. 7A, 7B and 7C illustrate example embodiments of web portals 1228, 1229 and 1231 respectively whereby a monitor 300 may customize ‘alert’ preferences for a given user or users 200. As reflected therein, these web portals 1228, 1229, 1231 contain functionality allowing the monitor 300 to: add/delete/edit monitor contact information; select for each monitor contact the circumstances (e.g. missed/failed breath tests, daily, weekly, monthly) and methods (e.g. text or email) in which the monitor contact associated with the monitor contact information will receive automated reports and/or alerts. Each of these functionalities, as well as the functionalities of other web portals may be implemented in part or in whole via monitor/user fillable fields, drop down menus and/or selectable icons 1226 and previously created contact fields 1230 which can be implemented via similar components. As shown in FIG. 7C, a confirmation screen can be shown in a web portal 1231.

As discussed with respect to the example embodiments, generated reports may include more or less information than what is described herein, but which is nonetheless apparent to one of ordinary skill in the art as desirable for effective sobriety monitoring. Accordingly, in some embodiments, the generated reports may comprise an alert, which can be a simplified report with limited information transmitted to the monitor 300 so that the monitor 300 may be apprised of an important event such as a missed/failed breath test by a user 200. An alert, for example, may include a text message that identifies the user 200 and the important event. Upon receiving the alert, the monitor 300 may use the monitoring station 1200 (or any of a variety of wired or mobile devices as described herein) to access the server supported website and review the full generated report having all the requested and/or provided monitoring information. As with the previously described reports, it is contemplated that the monitor 300 may customize preferences regarding the alerts not only with regards to the information provided, but as to how the alerts are transmitted and/or displayed.

In some circumstances, the monitor 300 may be responsible for monitoring a plurality of users. In such circumstances, the teachings described herein are applicable to the plurality of users. Further, the generated report may be a combined report, viewable via the server supported web site, containing hyperlinks or other access to the reports of each individual user 200.

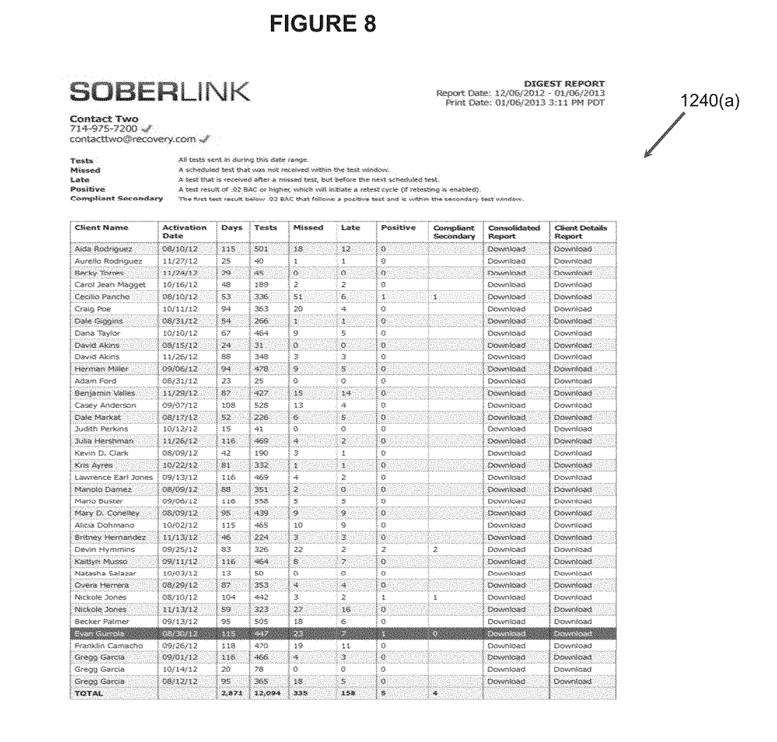

FIG. 8 illustrates an exemplary combined report 1240(a), comprising a table of monitored users 200 with fields for user (i.e. client) name, device activation date, number of days monitored, number of administer breath tests, number of missed/failed/late tests as well as compliant follow-up tests, and hyperlinks to downloadable reports for each user 200. In some embodiments, the combined report may indicate—via highlighting or other visual cue—where one or more monitored user(s) 200 have failed one or more administered breath tests, so as to draw the monitor's 300 attention to those user(s) 200.

Additional details of these features and others may be found in U.S. Pat. No. 8,707,758, issued on Apr. 29, 2014; U.S. Pat. No. 8,381,573, issued on Sep. 15, 2010; and U.S. application Ser. No. 13/274,553, filed on Oct. 17, 2011, the entire disclosures and contents of which are herein incorporated by reference in their entirety. Additional details of these features and others may also be found in the Appendix hereto filed herewith, the entire disclosure and content of which is herein incorporated by reference in its entirety.

### Additional Data Based Features

In addition to the breath test data, in various embodiments other data may also be transmitted to the monitoring station 1200, server 1700 and devices 1500 (and/or included in the report), including but not limited to: user-identification data (as further described herein), device location data, breath test timestamp data, anti-cheating data and/or device status data (including device connectivity, calibration and/or security data). Importantly, these categories of data types are supplied for illustrative purposes only, and it should be understood that the generated data referred to herein may belong one or more (or other) of these data types. It will be understood by one of ordinary skill in the art that wherever the transmission of breath test data is herein described, such descriptions may also apply to the transmission of other data described herein.

### User Verification Features

User identification data for a particular user 200 can include or consist of data utilized by the system to verify the identity of the user 200 taking a breath test using a particular breath monitoring device 1000. The user identification data may comprise one or more of: image data, video data, biometric data (e.g. fingerprint, DNA, retinal scan, etc. data), username/password entry data, or any other type of data that may be used to verify the identity of the user taking the breath test (see, e.g. FIG. 5). This verification is useful to make it more difficult for non-users to administer the scheduled breath test (to themselves) in lieu of the particular monitored user 200, i.e. to curtail cheating. Where the user identification data indicates that a non-user has administered the scheduled breath test in lieu of the user 200, the breath test report generated therefrom may identify the administered breath test as a ‘fail’ event. In some embodiments, this can trigger unique “attempted cheating” alerts.

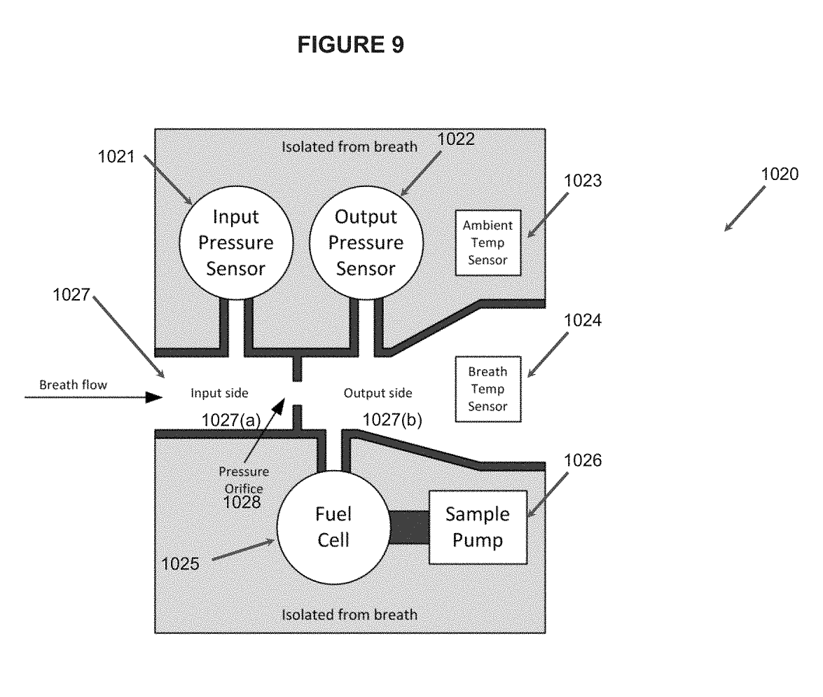

As reflected for example in FIG. 4, in some embodiments, a breath testing device 1000 comprises a test interface 1010 (see e.g. breath intake 1020 of FIG. 9), for receiving the user's breath during the scheduled breath test, and a camera or imager 1006 substantially adjacent the breath intake or otherwise positioned so as to capture an image of the user 200 during a breath test. Preferably, the camera 1006 is equipped with a lens capable of capturing the entire face of the user at close distances, such as a fish-eye lens. The captured image may be converted into image data to be transmitted to the monitoring station 1200, stored by server 1700 and/or utilized to verify the identity of the user taking the breath test.

Additional details of these features and others may be found in U.S. Pat. No. 8,707,758, issued on Apr. 29, 2014; U.S. Pat. No. 8,381,573, issued on Sep. 15, 2010; and U.S. application Ser. No. 13/274,553, filed on Oct. 17, 2011, the entire disclosures and contents of which are herein incorporated by reference in their entirety. Additional details of these features and others may also be found in the Appendix hereto filed herewith, the entire disclosure and content of which is herein incorporated by reference in its entirety.

### Other Anti-Cheating Features

In an example embodiment, anti-cheating data includes or consists of data verifying that the breath testing device 1000 (or the administered breath test itself) has not been interfered with in a way that would compromise the veracity of the breath test. The anti-cheating data may include or consist of breath characteristics data, component integrity data, image data, or any other type of data that may be used to verify that the breath testing device 1000 (or the administered breath test itself) has not been interfered with in a way that would compromise the veracity of the administered breath test. Where the anti-cheating data indicates the breath testing device 1000 (or the administered breath test itself) has been interfered with in a way that would compromise the veracity of the breath test, the breath test report generated therefrom may identify the administered breath test as a ‘fail’ event or, additionally or alternatively, as an ‘attempted cheating’ event.

### Breath Characteristic Data

In some embodiments, the breath testing device 1000 comprises one or more sensors for determining characteristics of the user's breath such as pressure (see e.g. FIG. 2 pressure sensor(s) 1404) and/or temperature (see e.g. FIG. 2 Temp sensor Breath+Ambient 1408), which may be utilized to generate breath characteristic data. The breath characteristic data may in turn be utilized to determine whether the breath testing device 1000 has been tampered or otherwise interfered with in a way that would compromise the breath testing data.

FIG. 9 illustrates a schematic diagram of an exemplary breath intake 1020 comprising: an input pressure sensor 1021, an output pressure sensor 1022, an ambient temperature sensor 1023, a breath temperature sensor 1024, a fuel cell 1025, a sample pump 1026 and a breath chamber 1027, including an input chamber 1027(a) partially separated from an output chamber 1027(b) by a pressure orifice 1028. As shown in FIG. 9, various components can be isolated or coupled with each other and/or breath areas as necessary. In an example embodiment, a user 200 breathes into input chamber 1027(a) and the breath leaves the device 1000 via output chamber 1027(b).

In some embodiments, the breath temperature sensor 1024 can detect the temperature of an airflow during an administered breath test, while an ambient temperature sensor 1023 can detect an ambient temperature of non-tested air, isolated from the breath flow. The pressure sensors 1021 and 1022 can detect the pressure of an airflow between the input side 1027(a) and the output side 1027(b) of chamber 1027 of breath applied during an administered breath test. Each of these components may be individually or collectively used to detect airflow tampering by a user 200.

As an example, human breath is generally no colder than thirty-three degrees Celsius, while compressed air from a container or other source may be significantly colder. Should a user 200 attempt to tamper with a scheduled breath test by ‘blowing’ into the breath testing device 1000 with compressed air, the breath temperature sensor may generate breath characteristic data indicating that the breath testing device has been tampered with accordingly. Moreover, the ambient temperature may be measured by ambient temperature sensor 1023 and used to further verify ‘human breath’ is being applied by comparing a measured temperature value with a temperature value measured by the breath temperature sensor 1024 using a processor and comparing with a threshold, range or other set of values to verify accuracy. For example, human respiration typically heats human breath temperature to at least fifteen degrees Celsius. If a measured ambient temperature is between ten and fifteen degrees Celsius, human respiration typically heats the breath temperature to at least twenty degrees Celsius. If the ambient temperature is above fifteen degrees Celsius, human respiration typically heats the breath temperature to at least twenty-eight degrees Celsius. When the ambient temperature is above thirty-three degrees Celsius, human respiration measured by the breath temperature sensor will typically be cooler than the ambient temperature. The temperature sensors 1023, 1024 may also be coupled with or include timers (not shown) detect time variances in temperature. Such time variances may indicate, for example, the presence of a heated air source that is heating up.

As an additional example, should a user block airflow from the output side 1027(b) of chamber 1027 to an exterior of the device 1000, the pressure in the output side 1027(b) of chamber 1027 would tend to increase relative to the pressure in the input side 1027(a) of the chamber 1027. The pressure sensors 1021, 1022 may detect this tampering and, when compared using a processor and programmed thresholds, ranges or other sets of values, generate breath characteristic data that indicates this tampering accordingly.

### Image Data—Blue Ring, Etc.

As reflected for example in FIG. 4, in some embodiments, a breath intake of a test interface 1010 can comprise structural components whose images are captured by a camera 1006 during a breath test to verify the veracity of the breath test.

In some embodiments, this can comprise a transparent mouthpiece 1010 into which a user 200 blows to administer a breath test. During the breath test, the mouthpiece 1010 may become more opaque upon exposure to the user's breath, an image of such transformation being captured by the camera 1006 and converted into image data to be transmitted to the monitoring station 1200, stored by server 1700 and/or utilized to determine whether the user 200 is faithfully performing the breath test, i.e. is not cheating. The mouthpiece 1010 may also comprise an indicator 1008, such as a ring, spot or other indicator, configured to illuminate during a breath test, such illumination to be captured by the camera 1006 during the breath test. In some embodiments, the indicator 1008 may be formed of a material that reacts to a light source on or within the breath testing device 1000, the light source activating during the scheduled breath test.

The image data may be transmitted to the monitoring station 1200, server 1700, and/or user devices 1500 in association with the breath test data and utilized by monitoring station 1200, server 1700, and/or user devices 1500 to confirm the veracity of the administered breath test. Alternatively, or additionally, the breath testing device may utilize the image data to confirm the veracity of the administered breath test.

In some embodiments the light source or indicator 1008 may change colors if a user passes or fails an administered breath test based on temperature, BrAC, pressure or other measurements.

### Breath Counting

In some embodiments, the breath testing device 1000 tracks a blow count reflecting the number of administered breath tests. The breath testing device 1000 generates blow count data from the tracked blow counts, and utilizes the blow count data to confirm the veracity of the administered breath test.

Accordingly, in some embodiments, the breath testing device 1000 may comprise a counter coupled to the breath testing module that registers a count during the administered breath test. In some embodiments, the breath testing module may comprise a pump (see, e.g. pump 1402 of FIG. 2) that engages when the air flowing through the pump 1402 exceeds a predetermined pressure. The counter may be coupled to the pump such that when the pump 1402 engages, the counter registers a count.

The monitoring station 1200, server 1700, and/or user devices 1500 may also track the number of received breath test data transmissions to compare against the blow count of the breath testing device 1000. The breath testing device 1000 may also transmit blow count data to the monitoring station 1200, server 1700, and/or user devices 1500 to be compared with the tracked number of received breath test data transmissions. In this manner, the monitor 300 may determine whether the cause of a missed breath test is because the breath test was not properly administered, or because the breath test data was not transmitted to the monitoring station 1200, server 1700, and/or user devices 1500 correctly.

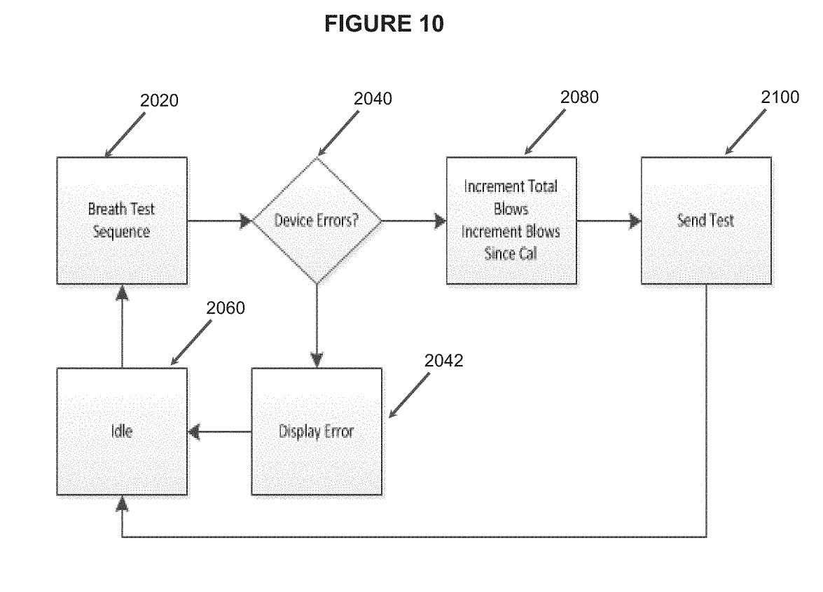

FIG. 10 illustrates an exemplary logic flow-chart for counter operation. At the initiation of the breath test sequence (Step 2020), the breath testing device checks for errors (Step 2040). If an error is detected, the device displays an error message (Step 2042) on the device display and then returns to an idle state (Step 2060). If no errors are detected, the counter is incremented (Step 2080), the breath test data is transmitted (Step 2100), and the device returns to the idle state (Step 2060). In some embodiments, the counter tracks the number of administered breath tests since the breath testing device 1000 was initiated, as well as the number of administered breath tests since the breath testing device 1000 was last calibrated. Accordingly, when the breath testing device 1000 is calibrated, the count value for the number of administered breath tests since the breath testing device 1000 was last calibrated may be reset.

In addition to monitoring the user 200, the blow count data may be utilized to track the reliability of the breath testing device 1000 for the purposes of maintenance, replacement or others.

### Timestamps

As described herein, in some embodiments timestamp data may be generated from a clock in association with an administered breath test using breath testing device 1000. The timestamp data reflects the time and/or date in which the breath test was administered, and may be transmitted to the monitoring station 1200, server 1700, and/or user devices 1500 in association with the administered breath test data.

In some embodiments, a monitoring station 1200, server 1700, and/or user devices 1500 operate(s) according to a default time, e.g. UTC time, while the breath testing device 1000 operates according to the clock time. In some embodiments, during a period of connectivity, a clock time may be reset to match the default time. In such circumstances, any timestamp data transmitted prior to the reset may be prorated to generate a modified timestamp reflecting the date/time of the administered breath test according to the default time. For example, when a periodic ‘check-in’ occurs and it is determined that the clock time is—for example—5 seconds behind the default time, the timestamp data for the associated breath tests transmitted at the ‘check-in’ is modified by that 5 second discrepancy to reflect the time of the administered breath test according to the default time. In this manner, the timestamp data for each administered breath test stored by the monitoring station 1200, server 1700, and/or user devices 1500 is according to a uniform date/time standard, e.g. the default time.

### Device Location Features

Device location data in some embodiments includes or consists of data utilized by the system to establish the geographical location of a breath testing device 1000. The device location data may comprise one or more of: global positioning system (“GPS”) data, Assisted GPS (“A-GPS”) data, and Advanced Forward Link Trilateration (“AFLT”) data. Many appropriate hardware/software components for generating the aforementioned device location data are known in the art. The device location data may be useful to verify the location of the user 200 during an administered breath test. Additionally, the device location data may be useful to verify the location of the breath testing device 1000 in the event it becomes lost or otherwise misplaced. Accordingly, the device location data may be transmitted to the monitoring station 1200, server 1700, and/or user devices 1500 independent of some or all breath test data, for example, during a periodic check-in with the monitoring station 1200, server 1700, and/or user devices 1500.

In some embodiments, location data is utilized by monitoring station 1200, server 1700, and/or user devices 1500 to record the geographic location of the breath testing device 1000 and in can be stored on monitoring station 1200, server 1700, and/or user devices 1500. The monitor 300 (and/or user 200) may access the location data via the interactive web portal, so as to track the location of the breath testing device 1000 at least when the device 1000 is administering a test. In this manner, the monitor 300 (and/or user 200) may determine the location of the breath testing device 1000. This feature may be useful to, for example, locate misplaced breath testing devices 1000, or track the location of the user 200, for example, if the user 200 is on house-arrest or some other form of detention.

Additional details of these features and others may be found in U.S. Pat. No. 8,707,758, issued on Apr. 29, 2014; U.S. Pat. No. 8,381,573, issued on Sep. 15, 2010; and U.S. application Ser. No. 13/274,553, filed on Oct. 17, 2011, the entire disclosures and contents of which are herein incorporated by reference in their entirety. Additional details of these features and others may also be found in the Appendix hereto filed herewith, the entire disclosure and content of which is herein incorporated by reference in its entirety.

### Device Status Features

Device status data can include or consist of data reflecting the operation status and parameters of the breath testing device 1000. As discussed herein, the breath testing device 1000 may periodically connect to a monitoring station 1200, server 1700, and/or user devices 1500 via the network 1300 so as to transmit—or otherwise exchange—data therewith. During this data transmission, the breath testing device 1000 may the transmit device status data to the monitoring station 1200, server 1700, and/or user devices 1500, which may include, for example, calibration data and/or security data.

In some embodiments, the periodic ‘check-in’ may occur according to predetermined time intervals. For example, the ‘check-in’ may coincide with the scheduled breath test. Alternatively, or in addition thereto, the ‘check-in’ may occur according to a schedule as, for example, every few hours, or minutes. In some embodiments, the periodic ‘check-in’ may occur every 5 minutes. However, if the ‘check-in’ fails, for example, due to failed network connectivity, the subsequent ‘check-in’ may be rescheduled according to one or more backup time intervals. For example, if the 5 minute check-in fails, the ‘check-in’ period may be reset to occur every 15 minutes, 30 minutes, or 60 minutes. In this way, the breath testing device 1000 may conserve battery life during extended periods of connectivity. In some embodiments, when connectivity is reestablished, the ‘check-in’ period may be automatically reset to the default period.

As discussed herein, during the ‘check-in,’ any data stored in device 1000 memory to be transmitted may be transmitted to a monitoring station 1200.

Additional details of these features and others may be found in U.S. Pat. No. 8,707,758, issued on Apr. 29, 2014; U.S. Pat. No. 8,381,573, issued on Sep. 15, 2010; and U.S. application Ser. No. 13/274,553, filed on Oct. 17, 2011, the entire disclosures and contents of which are herein incorporated by reference in their entirety. Additional details of these features and others may also be found in the Appendix hereto filed herewith, the entire disclosure and content of which is herein incorporated by reference in its entirety.

### Portable Calibration Station

It may be desirable that a breath testing device 100 be calibrated so as to maintain overall device 1000 and component reliability in some embodiments.

In many embodiments, device 1000 calibration may occur during a period of connectivity with s monitoring station 1200, e.g. the status check, as described herein. During device 1000 calibration, calibration data may be exchanged between the breath testing device 1000 and the monitoring station 1200 via wireless or wired communications. The calibration data may indicate whether the breath testing device 1000, and/or its component parts, is functioning as desired, as well as identify which—if any—errors are detected due to mis-calibration or tampering. The calibration data may also be utilized by the system to re-calibrate any mis-calibrated or tampered with components. For example, in some embodiments, a breath testing device clock may during calibration be reset to the time standard used by the monitoring station 1200, e.g. the current Coordinated Universal Time (“UTC”). System updates for the device 1000, such as software updates, may also be implemented during calibration.

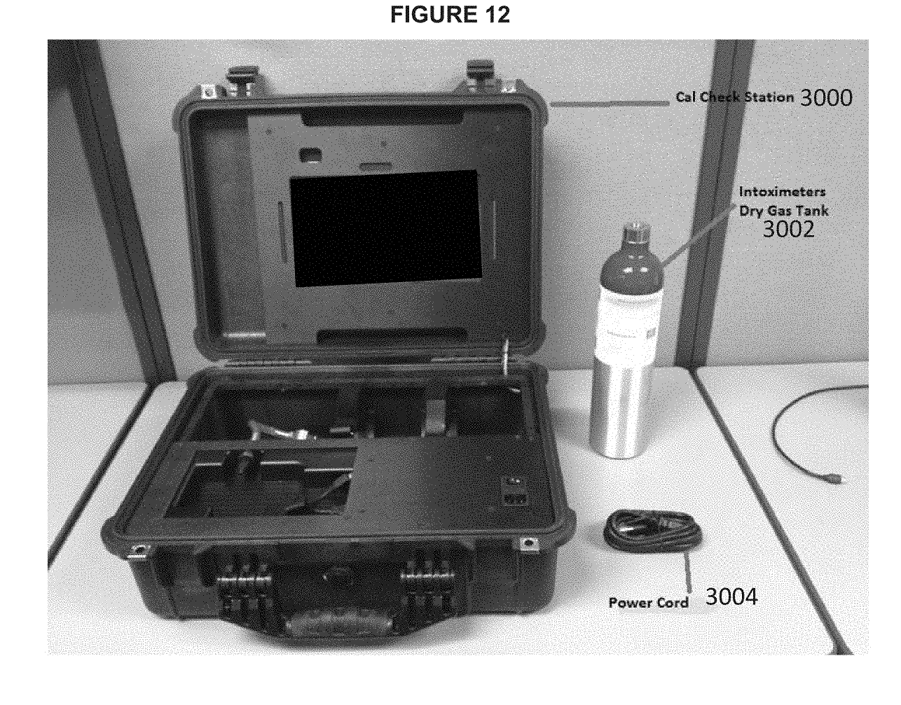

In some embodiments, device 1000 calibration may occur via a calibration station 3000, such as for example shown in FIG. 12, which may be used by a monitor 300 to perform breath testing device calibrations, i.e. to calibrate the breath testing device 1000. Preferably, the calibration station 3000 is a portable calibration station. Additional details of these features and others may be found in U.S. Pat. No. 8,707,758, issued on Apr. 29, 2014; U.S. Pat. No. 8,381,573, issued on Sep. 15, 2010; and U.S. application Ser. No. 13/274,553, filed on Oct. 17, 2011, the entire disclosures and contents of which are herein incorporated by reference in their entirety. Additional details of these features and others may also be found in the Appendix hereto filed herewith, the entire disclosure and content of which is herein incorporated by reference in its entirety.

### Bluetooth

In some embodiments, a breath testing device 1000 may transmit the breath test data to an intermediary device, such as cellular phone, which in turn transmits the breath test data to a monitoring station 1200 via a network 1300 as shown for example in FIG. 1. Accordingly, a local network may comprise a personal-area-network (“PAN”), and the transceiver may comprise a personal-area-network transceiver (e.g. transceiver 1600 of FIG. 2). This can be part of or separate from network 1300.

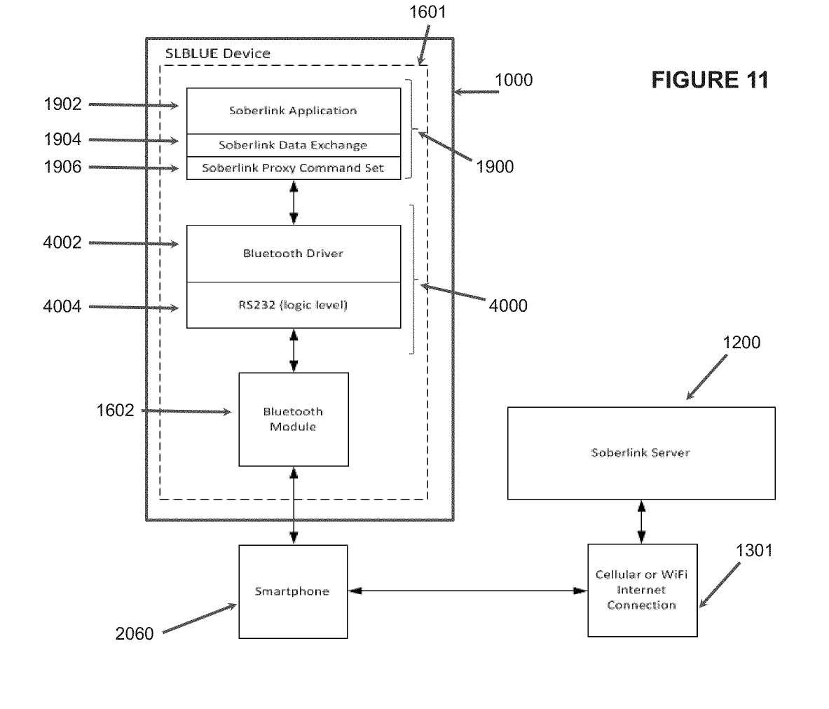

As shown for example in FIG. 11, a breath testing device 1000 may comprise a PAN module 1601 driven by a driver module and communicatively coupled to an intermediary device, such as a smartphone 2060. The intermediary device 2060 is in turn coupled to a monitoring station 1200 or other server 1700 via a wireless network over a cellular and/or Wi-Fi Internet Connection 1301. The PAN module 1601 and driver functionalities described herein may be controlled by a control unit (see e.g. processor 1800 of FIG. 2) executing software instructions retrievably stored in a non-transitory device memory.

In the example embodiment a device application 1902 can be stored in device memory and is associated with a data exchange module 1904 and proxy command set 1906 which combine to form a data module 1900. This data module 1900 can communicate with a Bluetooth driver 4002 and RS232 logic level module 4004 that combine to form a transmission control module 4000. Transmission control module can communicate via Bluetooth module 1602 which can include at least a Bluetooth transceiver 1602 communicating using Bluetooth protocol via connection with a Bluetooth transceiver of smartphone 2060 (not shown).

Additional details of these features and others may be found in U.S. Pat. No. 8,707,758, issued on Apr. 29, 2014; U.S. Pat. No. 8,381,573, issued on Sep. 15, 2010; and U.S. application Ser. No. 13/274,553, filed on Oct. 17, 2011, the entire disclosures and contents of which are herein incorporated by reference in their entirety. Additional details of these features and others may also be found in the Appendix hereto filed herewith, the entire disclosure and content of which is herein incorporated by reference in its entirety.

The enablements described in detail above are considered novel over the prior art of record and are considered critical to the operation of at least one aspect of the invention and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus, if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

Furthermore, the functionalities described herein may be implemented via hardware, software, firmware or any combination thereof, unless expressly indicated otherwise. If implemented in software, the functionalities may be stored as one or more instructions on a computer readable medium, including any available media accessible by a computer that can be used to store desired program code in the form of instructions, data structures or the like. Thus, certain aspects may comprise a computer program product for performing the operations presented herein, such computer program product comprising a computer readable medium having instructions stored thereon, the instructions being executable by one or more processors to perform the operations described herein. It will be appreciated that software or instructions may also be transmitted over a transmission medium as is known in the art. Further, modules and/or other appropriate means for performing the operations described herein may be utilized in implementing the functionalities described herein.

The scope of this description is to be interpreted only in conjunction with the appended claims and it is made clear, here, that the named inventor believes that the claimed subject matter is what is intended to be patented.

What is claimed is:

1. A system for a second user to remotely monitor the sobriety of a first user over a communications network, the system comprising:
a portable, cordless, handheld mobile breath testing device operable to receive the first user's breath and determine whether alcohol is present in the user's breath in excess of a predetermined quantity, said breath testing device including:
a portable, cordless hand-held housing,
a breath alcohol content sensor within the housing for sensing a breath alcohol content of the first user,
a wireless transceiver within the housing and communicatively coupled to a wireless network, the wireless transceiver transmitting breath test data generated based on the sensed breath alcohol content of the first user over the wireless network, wherein the wireless network is communicatively coupled to the communications network; and
a user interface device communicatively coupled to communications network, the user interface device operable to receive the transmitted breath test data and display the breath test data to the second user.

2. The system of claim 1, further comprising:
a server communicatively coupled to the communications network for receiving the breath test data and transmitting the breath test data to the user interface device.

3. The system of claim 2, wherein the server stores the breath test data.

4. The system of claim 1, wherein the device further comprises:
at least one pressure sensor for monitoring breath pressure during the first user's breath, and wherein the wireless transceiver transmits breath pressure data generated based on the sensed breath pressure of the first user over the wireless network for display to the second user.

5. The system of claim 1, wherein the device further comprises:
at least one temperature sensor for monitoring breath temperature during the first user's breath, and wherein the wireless transceiver transmits breath temperature data generated based on the sensed breath temperature of the first user over the wireless network for display to the second user.

6. The system of claim 1, wherein the device further comprises:
at least one pump coupled with at least one counter for monitoring breath pressure during the first user's breath and counting a number of breaths, and wherein the wireless transceiver transmits the number of breaths generated based on the sensed breath pressure of the first user over a predetermined period of time over the wireless network for display to the second user.

7. The system of claim 1, wherein the wireless transceiver transmits breath test data generated based on the sensed breath alcohol content of the first user over the wireless network to a smartphone coupled to the communications network and the smartphone forwards the breath test data to the user interface device via the communications network.

8. A method for a second user to remotely monitor the sobriety of a first user over a communications network, the method comprising:
a portable, cordless handheld mobile breath testing device including a processor coupled with a non-transitory memory storing instructions that, when executed by the processor, cause the processor to perform the steps of:
when a first user's breath is received via a breath receiving interface of the device, sense and determine whether alcohol is present in the user's breath by comparing a sensed amount of breath alcohol or lack thereof to a breath alcohol threshold to generate breath test data, and
transmit, via a wireless transceiver of the device that is communicatively coupled to a wireless network, the breath test data over the wireless network to a monitoring station operable to display the breath test data to a second user.

9. The method of claim 8, wherein the instructions further cause the processor to perform the following steps:
if the amount of breath alcohol meets or exceeds the threshold, trigger a failure user alert at the device, or
if the amount of breath alcohol does not exceed the threshold, trigger a passing user alert at the device.

10. The method of claim 9, wherein the alert is one or more of an audible user alert, a visual user alert, or a tactile user alert.

11. The method of claim 9, wherein the instructions further cause the processor to perform the following steps:
monitor whether an acknowledgement of the breath test data over the wireless network is received via the wireless transceiver from the monitoring station and, if no acknowledgement is received within a predetermined time, retransmit the breath test data over the wireless network.

12. The method of claim 9, wherein the instructions further cause the processor to perform the following steps:
when a first user's breath is received via a breath receiving interface of the device, capture an image of the first user for storage with the breath test data using an imager of the device, and
transmit, via the wireless transceiver, the image of the first user with the breath test data over the wireless network for display to the second user.

13. The method of claim 9, wherein the instructions further cause the processor to perform the following steps:
when a first user's breath is received via a breath receiving interface of the device, sense and determine a pressure of the user's breath by comparing a sensed breath pressure to a breath pressure threshold to generate breath pressure data, and
transmit, via the wireless transceiver, the breath pressure data with the breath test data over the wireless network for display to the second user.

14. The method of claim 9, wherein the instructions further cause the processor to perform the following steps:
when a first user's breath is received via a breath receiving interface of the device, sense and determine a temperature of the user's breath by comparing a sensed breath temperature to a breath temperature threshold to generate breath temperature data, and
transmit, via the wireless transceiver, the breath temperature data with the breath test data over the wireless network for display to the second user.

15. A device for a second user to monitor the sobriety of a first user, the device comprising:
a portable, hand-held housing;
a breath receiving interface;
a breath alcohol content sensor coupled with the breath receiving interface for sensing a breath alcohol content of the first user;
a processor, communicatively coupled with the breath alcohol content sensor and performing a breath alcohol analysis function on the sensed breath alcohol content for generating breath test data; and
a non-transitory computer readable memory for storing the breath test data.

16. The device of claim 15, further comprising:
a wireless transceiver operable to communicatively couple the device to a wireless network, wherein the wireless transceiver transmits the breath test data over the wireless network to a user interface device communicatively coupled to a communications network that is coupled to the wireless network, such that the breath test data can be displayed for the second user on the user interface device.

17. The device of claim 16, wherein the wireless transceiver is a Bluetooth transceiver.

18. The device of claim 15, wherein a breath testing module of the device comprises the breath alcohol content sensor.

19. The device of claim 18, wherein the breath testing module further comprises one or more of a pressure sensor, a pump and a temperature sensor.

20. The device of claim 15, wherein the device further comprises one or more of:
an audible alert module, a visual alert module, and a tactile alert module, wherein each alert module is communicatively coupled with the processor and operable to alert the first user of one or both of a breath test failure and a breath test pass based on the breath alcohol analysis performed by the processor.
